(19) United States
(12) Patent Application Publication
(10) Pub. No.: US 2008/0294128 A1
(43) Pub. Date: Nov. 27, 2008

(54) SURGICAL DRAIN SUPPORT HARNESS

(76) Inventor: Jennifer C. Richards, Centennial, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/127,502

(22) Filed: May 27, 2008

Related U.S. Application Data
(60) Provisional application No. 60/931,477, filed on May 24, 2007.

(51) Int. Cl. A61M 1/00

(52) U.S. Cl. 604/327

(57) ABSTRACT

Embodiments presented herein provide for a surgical drain support harness worn especially while showering that holds and secures post-surgical drain receptacles used for draining fluids from patients following surgery. In embodiments, the drain support harness includes a strap of webbing material which begins at the waist, ascends up and around the neck, and descends to the waist, and a belt, which encircles the waist of the wearer. The two straps can attach together to form a vest-like harness. In embodiments, multiple hook tape attachments are designed to receive pouches that hold the drain receptacles in place. The mesh pouches can have loop tape affixed that attach to the hook tape on the harness. The harness, in design and material, can be lightweight and can dry quickly after each use, reducing daily maintenance.

### CROSS-REFERENCES TO RELATED APPLICATIONS

This application claims the benefit of and priority to provisional patent application Ser. No. 60/931,477 filed on May 24, 2007 by the present inventor.

### COPYRIGHT NOTICE

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### BACKGROUND

After surgeries, the patient often experiences excessive fluid build-up near the surgery site because of extra lymph-node secretion or bleeding issues caused by the trauma of surgery. Surgeons place surgical drains to remove the excess fluid to prevent swelling and infection. Tubes are attached to the internal mechanism of the drain, placed in the body cavity at the surgical site and leave the body through an incision. The skin is stitched up around the tube to hold it in place. At the exterior end of the tube, a bulb (e.g., the most widely used Jackson-Pratt drain and Snyder drain) creates suction when the bulb is squeezed, removing fluid from the body.

Most often, patients are sent home with these drains and generally perform daily activities while trying to prevent pulling on the drainage tubes or dropping the drain bulbs. When the tubes move around or are tugged, the patient feels discomfort at the incision site—some patients even tear out sutures. Injuries caused by pulling on the drain tubes can require extra medical intervention, can cause pain, and can cause a longer recovery period. In the past, surgical drain receptacles were taped to a patient or pinned to undergarments. Because the bulbs have to be emptied on a regular basis, the adhesives on the tape can become less sticky, and, if people are sensitive to tape, the tape can cause major skin irritation. If safety pins are used, the time and energy it takes to attach and reattach the safety pins to undergarments can exhaust a post-surgical patient. The pins may also open accidentally and the drains can detach from the garment causing injury. Showering with the drain tubes can be especially difficult. The patient generally needs to secure the drains in order to free hands for washing. Pinning drains to some type of fabric creates the risks of detachment or being jabbed with the sharp pins. Garments for holding drain tubes often get saturated during bathing and sag or become heavy. The sagging garment often causes pulling on the drain tubes. Further, the garments restrict access to all of the skin for cleaning. Further, the waterlogged garments can become uncomfortable. The garments may also drip water on the floor and create a slipping hazard.

It is in light of these and other circumstances that the following application is being presented.

### SUMMARY

Embodiments presented herein are generally related to a harness for holding drain tubes or other medical devices. In embodiments, the harness is worn in a vest-like manner, having a strap of webbing material that begins at the waist of the wearer, travels up the abdomen and chest, loops around the neck, and descends down the other side of the chest and abdomen, until it reaches the waist. The ends of the strap can be attached to a belt strap made of the same webbing material that encircles the waist of the wearer. The ends of the straps can be connected to the straps by hook and loop fasteners. One or more holders can be connected or attached to the straps of the harness. Each pouch may be adapted in size and shape so as to receive and maintain therein one or more drainage receptacle.

### BRIEF DESCRIPTION OF THE DRAWINGS

The disclosure will be more readily understood from a reading of the following specification and by reference to the accompanying drawings forming a part thereof, wherein an example of the disclosure is shown and wherein:

FIG. 1A is a three-dimensional illustration of a drain support harness as it is worn by an individual;

FIG. 1B is another three-dimensional illustration of a drain support harness;

FIG. 1C is yet another three-dimensional illustration of a drain support harness;

FIG. 1D is a perspective drawing of an embodiment a drain support harness viewed from above;

FIG. 1E is a perspective drawing of an embodiment of a drain support harness viewed from the front;

FIG. 1F is a perspective drawing of an embodiment of a drain support harness viewed from the side;

FIG. 1G is a perspective drawing of another embodiment of a drain support harness viewed from the front;

FIG. 1H is a perspective drawing of another embodiment of a drain support harness viewed from the front;

FIG. 2 is a flowchart of an embodiment of a method for manufacturing the drain support harness;

FIG. 3 is a flowchart of an embodiment of a method for manufacturing the pouches to be attached to the drain support harness; and

FIG. 4 is a flowchart of an embodiment of a method for wearing of the drain support harness by an individual.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

Embodiments presented herein provide a surgical drain support harness for holding one or more surgical drains. In one embodiment, a patient wears a first strap around his or her waist similar to a belt. A neck strap is attached substantially perpendicular to the belt strap by one end. The neck strap is worn around the neck and attaches substantially perpendicular to the belt strap at a second end. Thus, the weight of the harness and supported drain tubes are held by the neck of the patient. One or more drain tube holders are attached to either the belt strap or the neck strap.

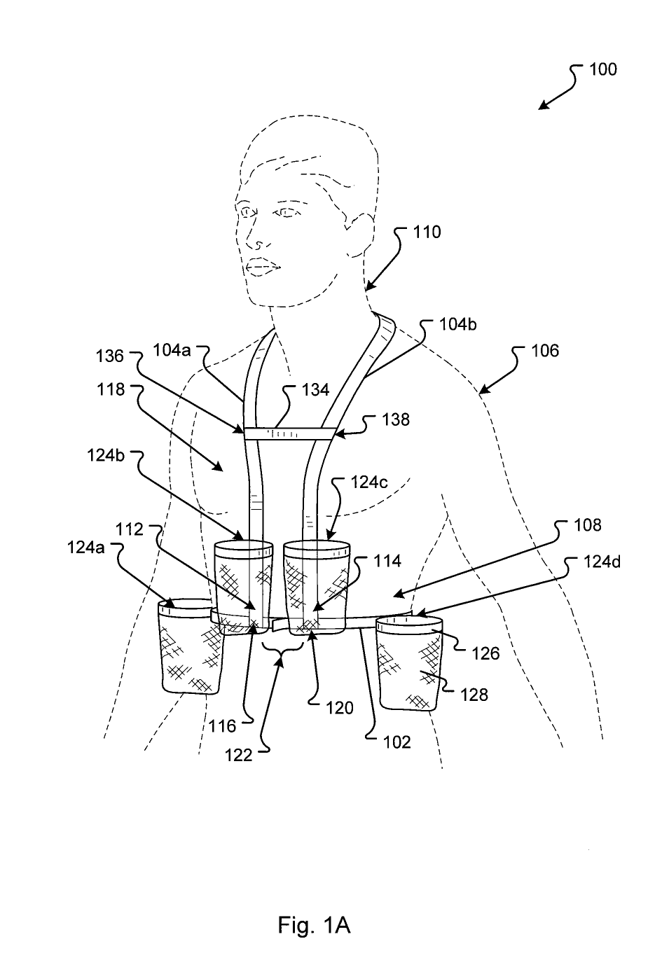

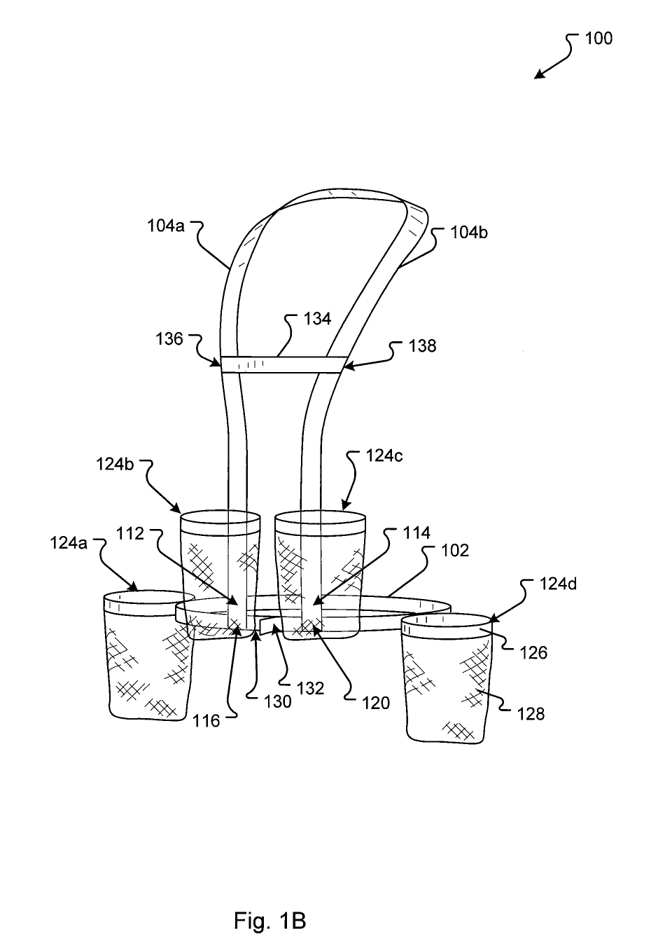

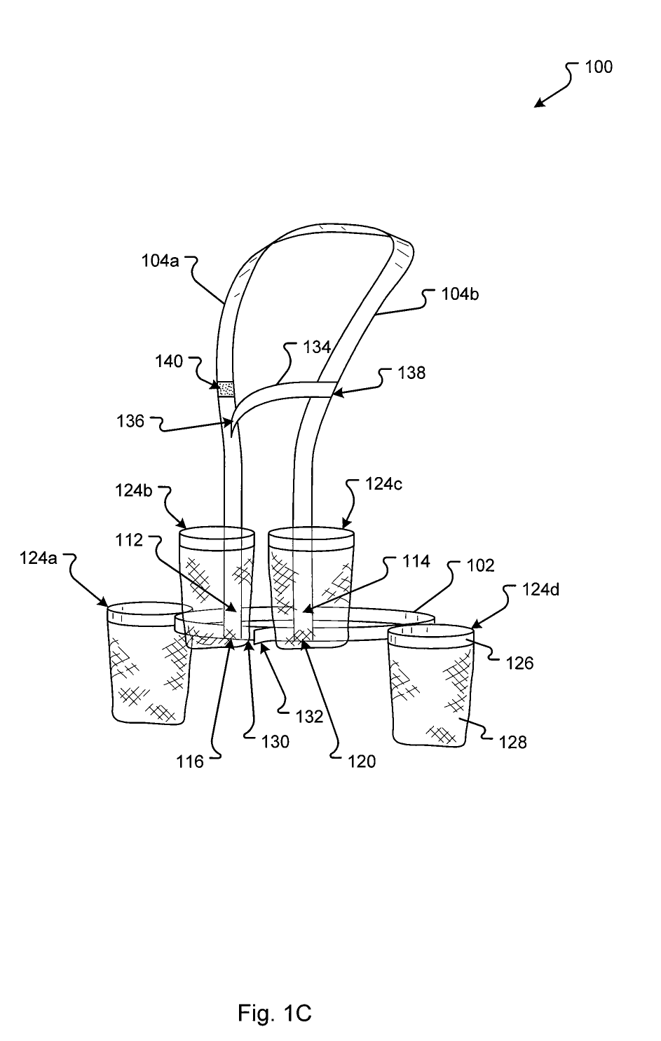

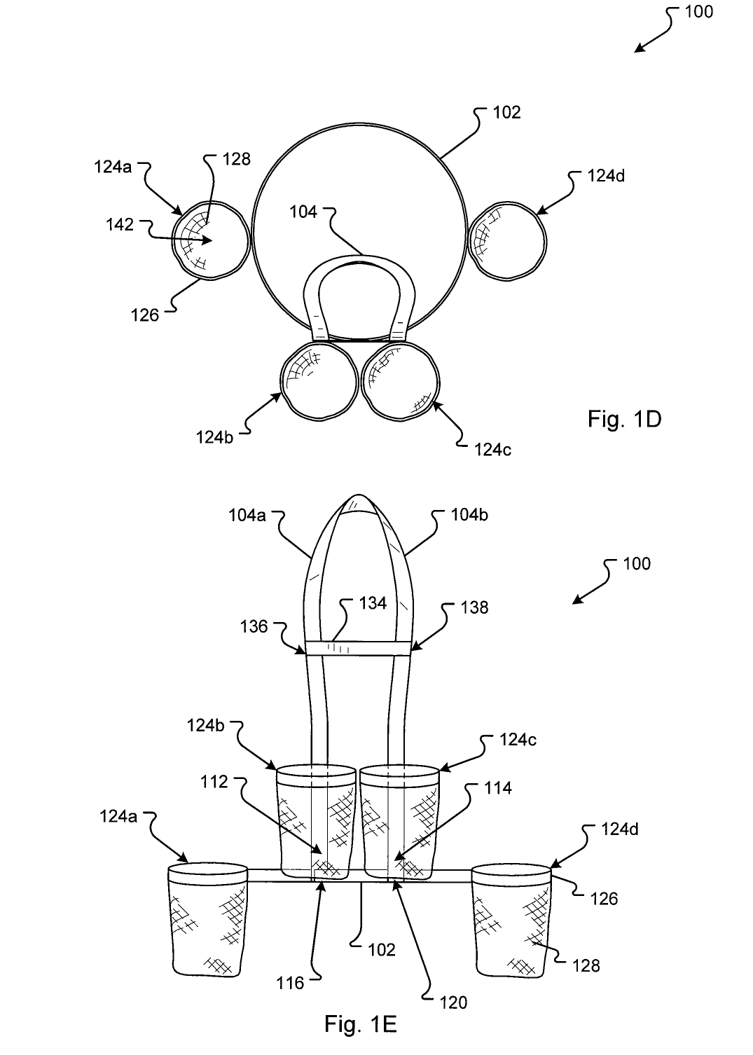

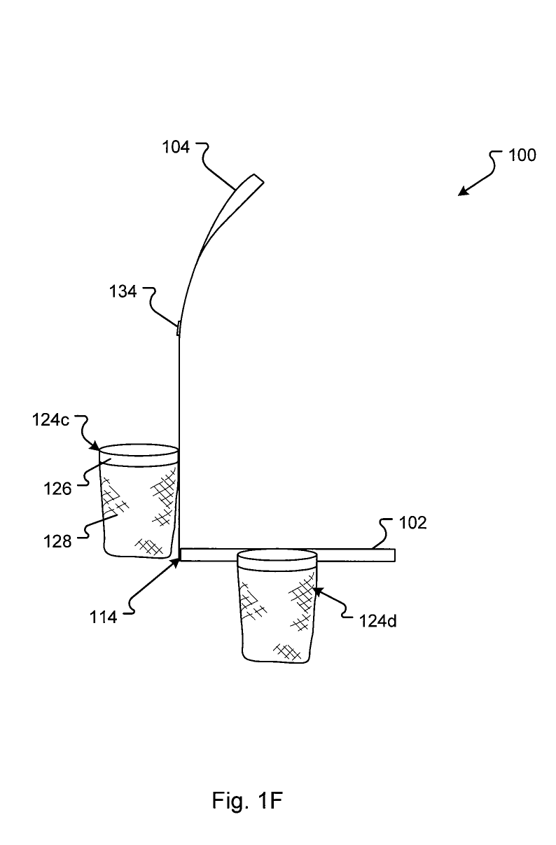

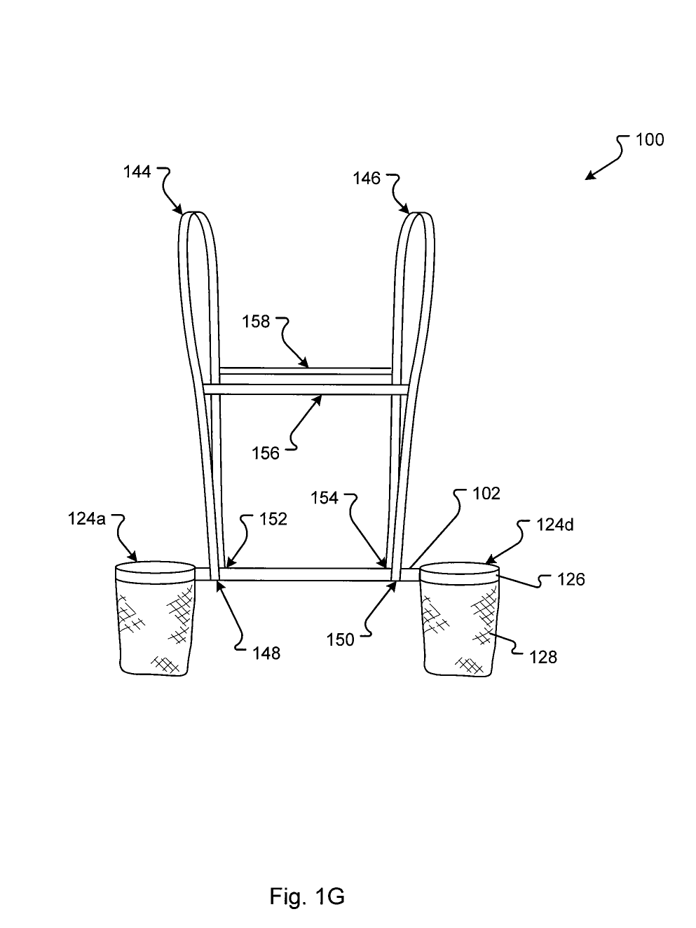

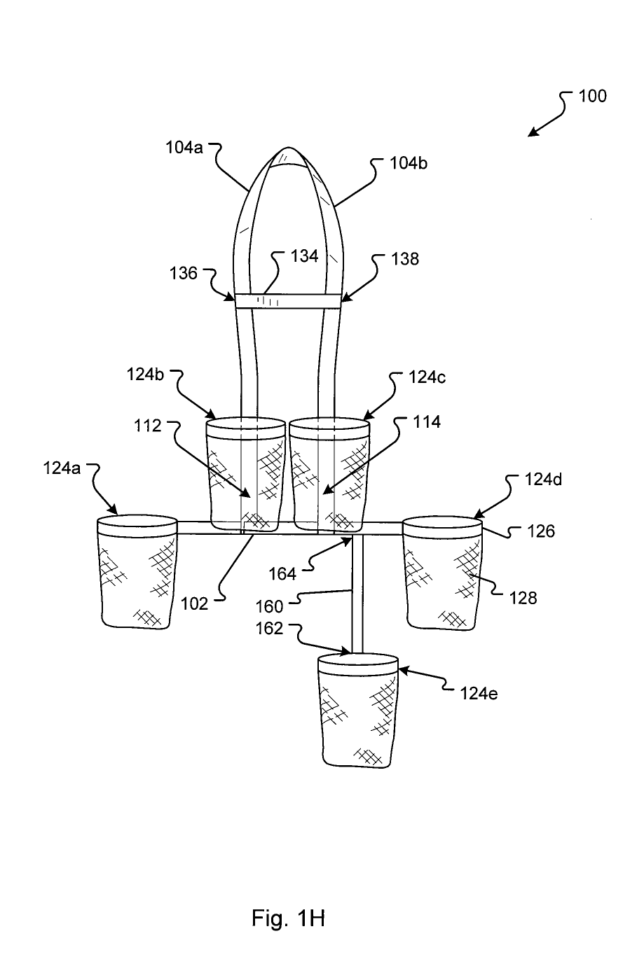

One or more embodiments of a harness 100 are shown in FIGS. 1A-1H. The harness 100 will be explained with references to each of the FIGS. 1A-1H, wherein like reference numbers refer to the same item in each of the figures. In embodiments, the harness 100 includes a first member 102 and one or more second members 104. The first member is worn or held circumferentially around the torso 108, or waist, of the patient 106, as shown in FIG. 1A. In other words, the first member 102 may be worn as a belt. As such, the first member 102 may be referred to as a “belt strap” or, simply, as a “belt”. The second member 104, in one embodiment, is worn around the back, or posterior, of the neck 110 of the patient 106, as shown in FIG. 1A. As such, the second member 104 may be referred to as a “neck strap” or “loop strap” because of the loop formed in the second member 104.

In embodiments, each member 102 and/or 104 and other parts of the harness 100 can be connected, attached, coupled, or bonded together. One skilled in the art will recognize various methods for connecting, attaching, coupling, or bonding the parts together and such attachments are not confined to any one method. In an embodiment, a piece of loop tape is affixed to the interior facing portion of a second end of the belt strap 102. The loop tape corresponds with a piece of hook tape that is attached to the exterior portion of the first end of the belt strap 102. The opposite ends of the bottom strap 102 can detachably connect and open with ease, forming the first member 102 of the harness 100 (FIG. 1). In another example, the second member 104 is detachably connected or connected to the first member 102 using a hook and loop fastener, such as Velcro®. In other embodiments, the first member 102, the second member 104, and/or other parts of the harness 100 are detachably connected with buttons, snaps, adhesives, stitching, etc. Regardless of the methods or means of attaching the parts of the harness 100 together, the harness 100 may have several configurations. One configuration is shown in FIGS. 1A-1F.

A first configuration has the belt strap 102 and the neck strap 104. The first member 102 can have a first end 130 and a second end 132. The first end 130 and the second end 132 may be detachably connected to each other using a connector, such as a hook and loop fastener. Thus, the patient 106 can detach the first end 130 and the second end 132, slip the first member 102 around the patient's waist, and reattached the first end 130 to the second end 132. In embodiments, the second member 104 also has a first end 112 and a second end 114. The first end 112 can be connected with the first member 102 at point 116, substantially perpendicular to both the belt strap and the chest strap as shown in FIGS. 1A-1F. The second member 104a extends from point 116 superiorly along the anterior portion 118 of the patient 106. In other words, the second member 104a can begin at the waist of a wearer 106 and ascend up the chest of the wearer 106. The second member 104 is then held circumferentially along the posterior of a neck 110 of the patient, that is, the second member is worn around the neck 110. Then, the second member 104b can extend inferiorly from the neck 110 of the patient 106 back to the first member 102 (i.e., descends back down the chest). The second end 114 of the second member 104b can also be attached to the first member 102, at a second point 120, which may be some predetermined distance 122 from the first point 116.

Each member 102 and/or 104 can be made of different materials. In one embodiment, the members 102 and/or 104 are made from polypropylene webbing, other polypropylene material, nylon, or other materials. As such, the members 102 and/or 104 may be referred to as “straps”. However, the members 102 and/or 104 can be made from rigid materials, other fabrics, or other materials that can function to create the harness 100 substantially as described and shown. Hereinafter, the members 102 and/or 104 will be described as straps but this terminology is not meant to limit the members 102 and/or 104 to those particular embodiments.

In embodiments, the harness 100 also includes one or more holders 124a, 124b, 124c, and/or 124d. The holders 124a, 124b, 124c, and/or 124d may also be referred to as containers. The holders 124a, 124b, 124c, and/or 124d can be formed at a first end from an attachment member 126, which attaches to either of the members 102 and/or 104, and a pouch 128. The attachment member 126, in embodiments, is formed as to create an opening 142, substantially as shown in FIG. 1D. The size of the opening 142 and the pouch 128 can be formed to accept and/or hold a container for a surgical drain receptacle. The pouch 128 can comprise a front, one or more sides, and a rear (pouch 128 sidewalls can be stitched together along a side seam, a bottom seam, and along the attachment with the attaching member 126 to create the interior of the pouch 128). The sides at the top of the pouch 128 can form a lip at the top of the pouch 128. In embodiments, the attachment member 126 is formed from a piece of twill tape, folded in half, and accepting the lip of the pouch 128. The lip of the pouch 128 can be stitched to the twill tape, creating a finished lining to the pouch lip and creating the attachment member 126. In embodiments, the pouch 128 is made from a webbed material or webbing that prevents the accumulation of water in the pouch during showering with the harness 100.

Due to the construction of one or more embodiments of the holders 124a, 124b, 124c, and/or 124d, the holders 124a, 124b, 124c, and/or 124d may also be referred to hereinafter simply as “pouches”. However, the holders 124a, 124b, 124c, and/or 124d may have one or more other designs. For example, the pouch 128 may be formed from a cylinder of predetermined length. The cylinder may have a bottom. The bottom of the cylinder can have one or more openings to allow water to escape the cylinder. The other end of the cylinder may be left open to accept the surgical drain receptacle. The holders 124a, 124b, 124c, and/or 124d can also be formed from bowls, bags, or other constructions, as will be evident to one skilled in the art. Each of the holders 124a, 124b, 124c, and/or 124d may hold one or more surgical drain receptacles while the harness 100 is used by the patient 106.

In one embodiment, on the backside of the pouch 124a, 124b, 124c, and/or 124d, a piece of loop tape is attached to the pouch lip 126 on the twill tape. This piece of loop tape corresponds to and can connect to the pieces of hook tape on the loop strap 104 and/or belt strap 106. The pouches 124a, 124b, 124c, and/or 124d can be held in an upright position and may be deep enough to keep the drain bulbs completely secure in the pouch 124a, 124b, 124c, and/or 124d. Because there are numerous areas for pouches 124a, 124b, 124c, and/or 124d to attach to the surgical drain support harness 100, the wearer can place the pouch 124a, 124b, 124c, and/or 124d where most comfortable, e.g. at chest height or around the waist.

In alternative embodiments, the harness 100 also comprises a third member 134. The third member 134, in embodiments, includes a first end 136 and a second end 138. The first end 136 can be attached to the second member 104a as the second member 104a extends superiorly between the first end 112 and the neck 110 of the patient 106, substantially parallel to the belt strap as shown in FIG. 1A. The second end 138 can be attached substantially parallel to the belt strap but substantially perpendicular to the second member 104b as the second member 104b extends inferiorly between the neck 110 of the patient 106 and the second end 114 of the second member 104b. Hereinafter, the third member 134 may be referred to as a “chest strap” or “cross strap,” however, this term should not limit the possible configurations or placements of the third member 134.

In embodiments, the chest strap 134 is placed approximately two-thirds up the loop strap 104, on the exterior facing side of the loop strap 104. The chest strap 134 may be between five and ten inches long. The second end 138 of the cross strap 134 is sewn to the loop strap 104. The first end 136 of the cross strap 134 contains a piece of small loop tape, attached on the interior facing side of the first end 136. This loop tape detachably connects with a corresponding piece of hook tape 140 affixed to the loop strap 104 in the same vertical location where the first end 136 touches or contacts the second member 104. The hook tape 140, in embodiments, is placed on the exterior side of the loop strap 104, substantially as shown in FIG. 1C. This allows the cross strap 134 to open and close with ease and makes the harness 100 easy to put on. The cross strap 134 also keeps the support harness 100 secure when one or more other hook and loop fasteners, on the harness 100, are opened to wash the skin underneath the straps 102 and/or 104. Thus, the embodiments presented herein have the advantage of allowing the patient 106 to easily access the skin under the harness 100 for washing.

In embodiments, the materials used for the members 102 and/or 104, the holders 124a, 124b, 124c, and/or 124d, and or the chest strap 134 are waterproof or water resistant. As such, the harness 100 can be worn in the shower without getting substantially heavier either by absorbing water or collecting water in the holders 124a, 124b, 124c, and/or 124d. In embodiments, the holders 124a, 124b, 124c, and/or 124d are made of aquatic mesh material to prevent retaining water, as sold by Collins Cottage Industries located in Church Hill, Tenn. In alternative embodiments, the exterior faces of the members 102 and/or 104 and/or the chest strap 134 are covered in either hook tape or loop tape. The attachment members 126 of the holders 124a, 124b, 124c, and/or 124d are covered in the corresponding hook tape or loop tape. As such, the holders 124a, 124b, 124c, and/or 124d may be detachably connected to the harness 100, with the hook and loop fastener, in any position and not just the positions shown in FIGS. 1A-1H. In other embodiments, the hook and loop fasteners are replaced with buttons, snaps, adhesive, a hanger, or other device to allow for the many configurations possible with the holders 124a, 124b, 124c, and/or 124d and the harness 100.

The harness 100 may have several different configurations beyond the embodiment shown in FIGS. 1A-1H. The harness 100 may exclude the belt strap. In other embodiments, the loop strap is replaced by one or more straps that go over the shoulder of the patient 106 and attach substantially perpendicular to the belt strap at the anterior and posterior of the patient 106. For example, an embodiment of the harness 100 is shown in FIG. 1G in which two straps 144 and/or 146 each go over the shoulder of the patient. Herein, a first end 148 and/or 150 of the shoulder straps 144 and/or 146 is attached substantially perpendicular to the anterior of the belt strap 102. A second end 152 and/or 154 of the shoulder straps 144 and/or 146 are attached to the posterior of the belt strap 102. The configuration allows the patient 106 to wear the shoulder straps 144 and/or 146 over the shoulders. One or more holders 124a and/or 124d may be detachably connected to the harness 100 at various positions on the belt strap 102 and/or the shoulder straps 144 and/or 146. In embodiments, the shoulder straps 144 and/or 146 may also be connected by one or more chest straps 156 and/or 158. Other configurations of the straps are possible. For example, there may be only one shoulder strap. The single shoulder strap may be worn at an angle across the torso from one shoulder and connecting to the belt strap at the opposite hip (similar to a crossing guard uniform). In other embodiments, the two straps may be crossed either at the anterior of the torso or at the posterior of the torso or at both the anterior and posterior of the torso. The different configurations can be created to fit the needs of the patient 106 according to what medical equipment the patient 106 may need to hold in the harness 100. One skilled in the art will recognize other configurations that are contemplated within the scope of disclosure.

Another embodiment of the harness is shown in FIG. 1H. Herein, an extension strap 160 is attached, at a first end 164, and extended substantially perpendicular from a portion of the harness 100, in this embodiment, from the belt strap 102. The extension strap 160 can support a holder 124e attached at a second end 162 or some other point on the extension strap 160. The extension strap 160 allows holder 124e to be used at different height to accommodate different surgical drains. For example, a drain placed in the leg may need to be held lower on the leg rather than around the waist, the extension strap having a first end detachably connect to the first strap or the second strap, the second end attached to a container.

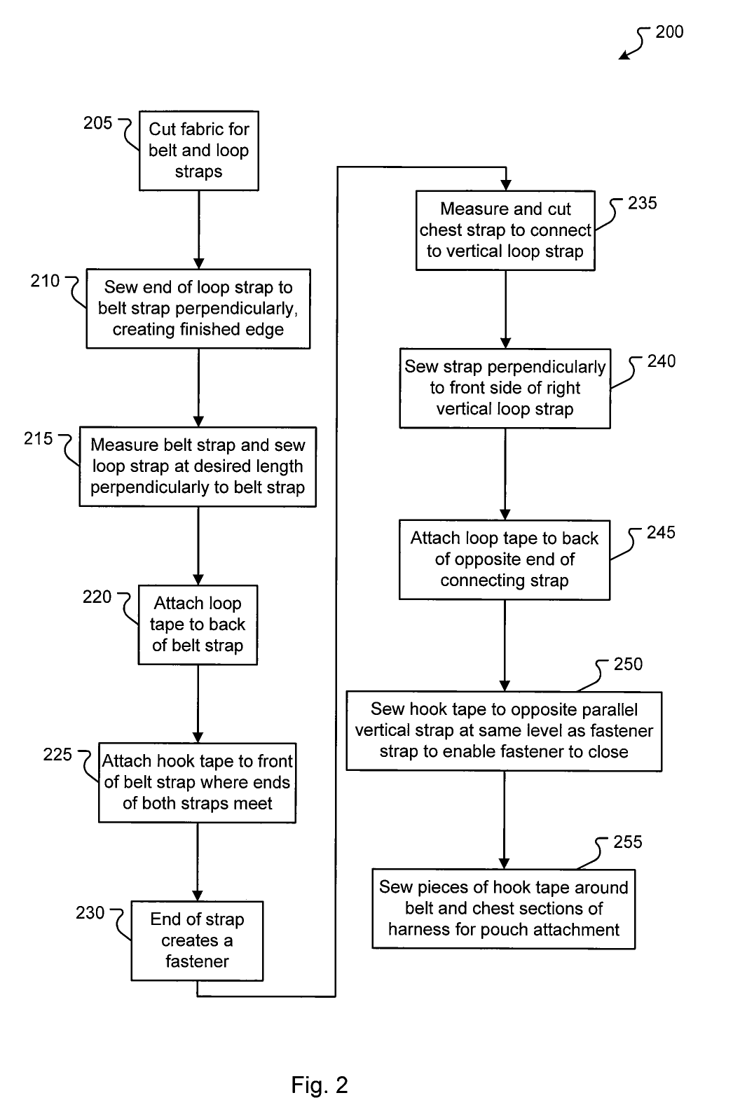

An embodiment of a method 200 for constructing a harness 100 (FIG. 1) is shown in FIG. 2. The steps shown in the method 200 may be performed by machinery operable to create a harness 100 (FIG. 1). Cut operation 205 cuts fabric or other material for the straps 102, 104 (FIG. 1), or other straps in the harness 100 (FIG. 1). The amount of material cut can correspond to the length and width of the straps. Sew operation 210 sews at least one end of the loop strap 104 (FIG. 1) to the belt strap 102 (FIG. 1) in a substantially perpendicular orientation. The sew operation 210 can create a finished end (i.e., an end that will not fray and may be pleasing to the eye and touch) to the end of the loop strap 104 (FIG. 1) attached to the belt strap 102 (FIG. 1).

Measure operation 215 can measure the belt strap 102 (FIG. 1) to locate where the first end of the loop strap 104 (FIG. 1) should be attached to the belt strap 102 (FIG. 1). In addition, the measure operation 215 may also include sewing the loop strap 104 (FIG. 1) to the location measured in the measure operation 215. Attach operation 220 attaches one or more pieces of loop tape to the backside of one end of the belt strap 102 (FIG. 1). Attach operation 225 attaches hook tape to the front-side of the opposite end of the belt strap 102 (FIG. 1). As such, the belt strap 102 (FIG. 1) can be detachably connected with the hook and loop fastener, as shown in end operation 230.

Measure operation 235 measures the length of the chest strap 134 (FIG. 1) to connect the chest strap 134 (FIG. 1) to the loop strap 104 (FIG. 1). The measure operation 235 may also including measuring the loop strap 104 (FIG. 1) to locate the chest strap 134 (FIG. 1) in the correct location on the loop strap 104 (FIG. 1). Sew operation 240 sews the chest strap 134 (FIG. 1) at one end substantially perpendicular to one portion of the loop strap 104 (FIG. 1). Attach operation 245 attaches loop tape to the back of the opposite end of the chest strap 134 (FIG. 1). Sew operation 250 sews hook tape to the opposite portion of the loop strap 104 (FIG. 1) to enable the chest strap 134 (FIG. 1) to be detachably connected, at one end, to the loop strap 104 (FIG. 1) using the hook and loop fastener. Sew operation 255 sews one or more pieces of hook tape to one or more portions of the harness 100 (FIG. 1) to allow for holders to be connected to the harness 100 (FIG. 1).

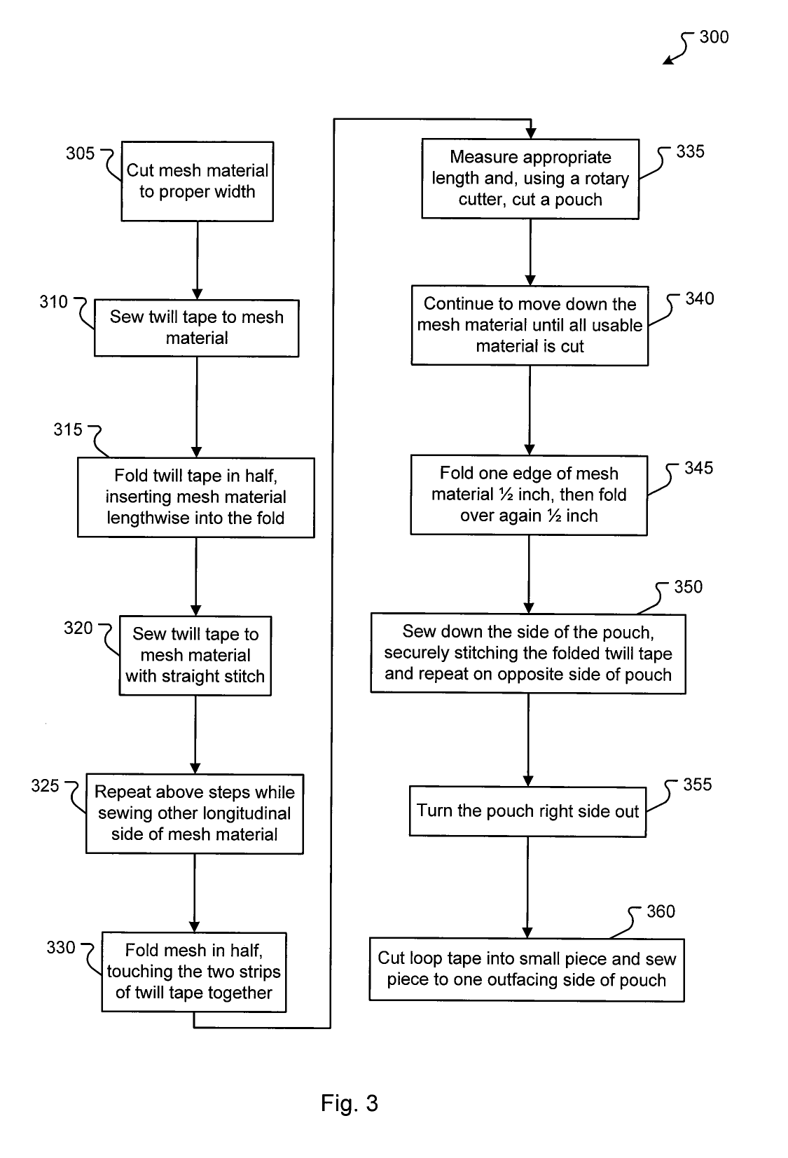

An embodiment of a method 300 for constructing a holder 124 (FIG. 1) is shown in FIG. 3. The steps shown in the method 300 may be performed by machinery operable to create a holder 124 (FIG. 1). Cut operation 305 can measure and cut a mesh material and/or twill tape to the proper height and width. Sew operation 310 sews the twill tape to one end of the mesh material. Fold operation 315 folds the twill tape over the end of the mesh material, inserting the mesh material lengthwise into the fold, and encompassing the end of the mesh material. Sew operation 320 sews the folded ends of the twill tape together with a straight stitch to create the attaching member. Repeat operation 325 will repeat the steps of attaching the twill tape to one or more other sides of the mesh material. Reinforcing the ends of the mesh with twill tape ensures the mesh material will not tear or fray and the twill tape provides a better surface to sew the portions of the pouch 124 (FIG. 1) together.

Fold operation 330 folds the mesh in half to have two strips of the twill tape touch. Measure operation 335 measures the appropriate length of the pouch 124 (FIG. 1) and cuts the pouch 124 (FIG. 1) to length. Continue operation 340 cuts mesh material to create the proper length for the pouch 124 (FIG. 1). Fold operation 345 folds a ½ inch portion of the mesh material twice. Sew operation 350 sews along the folded mesh material and along the end of the twill tape. The sew operation 350 may be repeated on all necessary sides of the pouch 124 (FIG. 1). Turn operation 355 turns or inverts the pouch 124 (FIG. 1) right-side out (which conceals the stitching). Cut operation 360 cuts and sews loop tape to the posterior side of the pouch 124 (FIG. 1) which can detachably connect to the hook tape sewn on the harness 100 (FIG. 1).

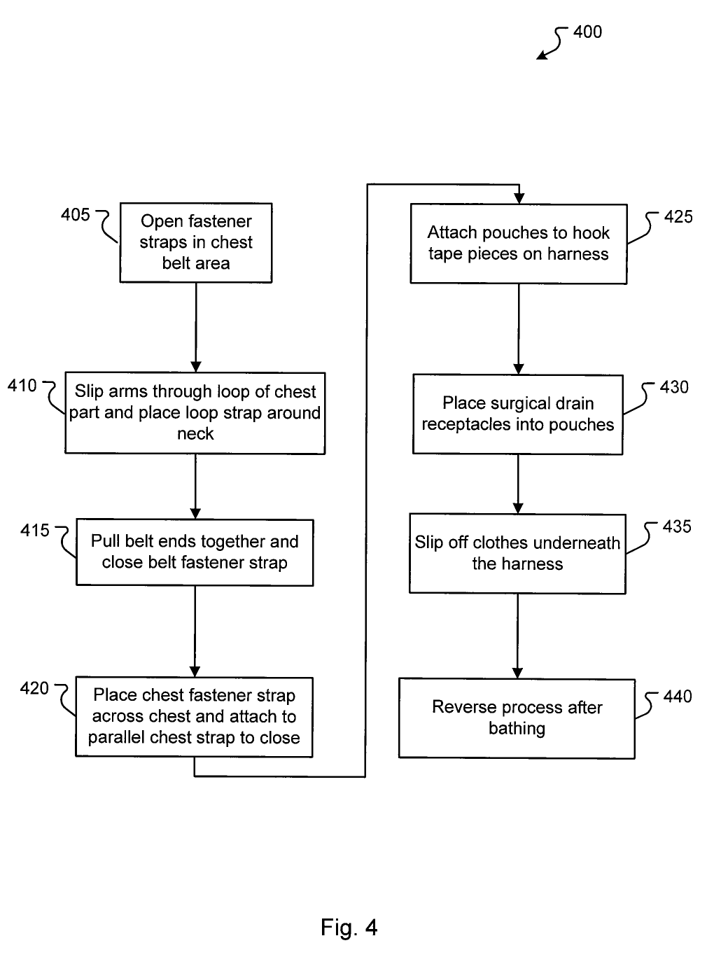

An embodiment of a method 400 for donning the harness 100 (FIG. 1) is shown in FIG. 4. The operations or steps of the method 400 may be performed by a person trying to don the harness 100 (FIG. 1). At step 405, a person opens a fastener for the belt strap 102 (FIG. 1), chest strap 134 (FIG. 1), and/or loop strap 104 (FIG. 1). At step 410, the person slips head through the loop strap 104 (FIG. 1). The person, at step 415, pulls the belt around the torso of the person and pulls the ends of the belt strap 102 (FIG. 1) together to close the belt strap 102 (FIG. 1). At step 420, the person places the chest strap 134 (FIG. 1) across the person's chest and attaches the chest strap 134 (FIG. 1) end to the loop strap 104 (FIG. 1). The person, at step 425 can then attach one or more pouches 124 (FIG. 1) to the hook tape on the harness 100 (FIG. 1).

If the person is a patient 106 (FIG. 1), the patient can, at step 430, place one or more drain receptacles into one or more pouches 124 (FIG. 1). At step 435, the patient may then take off his or her clothes underneath the harness 100 (FIG. 1) and bathe. After bathing, the patient 106 (FIG. 1), at step 440, can reverse the steps 405-435 to take off the harness 100 (FIG. 1).

While various aspects of embodiments of the disclosure have been summarized above, the detailed description illustrates exemplary embodiments in further detail to enable one of skill in the art to practice the disclosure. In the description, for the purposes of explanation, numerous specific details were set forth in order to provide a thorough understanding of the present disclosure. It will be apparent, however, to one skilled in the art that the present disclosure may be practiced without some of these specific details. Several embodiments of the disclosure are described, and while various features are ascribed to different embodiments, it should be appreciated that the features described with respect to one embodiment may be incorporated with another embodiment as well. By the same token, however, no single feature or features of any described embodiment should be considered essential to the disclosure, as other embodiments of the disclosure may omit such features.

Also, it is noted that the embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in the figure.

The embodiments presented herein provide several advantages. For example, the harness 100 (FIG. 1) enables drain receptacles to be worn in multiple locations, wherein previous garments allowed holding the drain receptacles only in one or two set positions. Further, the harness 100 (FIG. 1) eliminates the need for pins or tape. The harness 100 (FIG. 1) can support one or more drainage receptacles securely simply by adding more holders 124 (FIG. 1). The harness 100 (FIG. 1) also minimizes the risk of drainage receptacles becoming detached from the surgical site.

Still further, the harness 100 (FIG. 1) provides full access to ones body for washing purposes, while still supporting drain receptacles in a secure fashion. When the harness 100 (FIG. 1) is worn in the shower, the drain tube bulb is placed in the pouch where it is securely held while the wearer's hands are free to wash. The fasteners can be opened, one at a time, to wash. The remaining fasteners stay closed to secure the harness while the skin area is washed under the opened fastener. The entire body can be accessed under the harness while all drainage receptacles remain fully secure in place.

Another advantage is that, since the weight of the harness belt 102 (FIG. 1) is supported by the loop strap 104 (FIG. 1) that goes around the wearer's neck, the belt strap 102 (FIG. 1) will not come undone and slip to the floor. Further, the harness 100 (FIG. 1) can be made of a light-weight polypropylene webbing which dries quickly and does not become waterlogged. The pouches 124 (FIG. 1) can be made of mesh and the water flows freely from the pouch and, thus, does not fill the pouches, which makes the harness 100 (FIG. 1) more comfortable, safer, and less messy to wear. Thus, the harness 100 (FIG. 1) solves the problems faced in previous garments by substantially preventing detachment and dislodgement of the drainage tube from the body without discomfort to the patient 106 (FIG. 1) while allowing complete access to the entire body for washing in the shower and preventing injury, mess, and discomfort from soaked garments.

Several modifications to the harness 100 (FIG. 1) are possible. For example, the harness 100 (FIG. 1) may be made of alternative materials, e.g. light-weight polypropylene webbing, mesh, nylon, cotton, polyester, rubber, lightweight plastic, and crocheted or braided yarn. Additionally, the pouches 124 (FIG. 1) may be made of any of these materials in such a way that appropriate drainage holes are provided to prevent the build-up or accumulation of water. In an embodiment, the harness 100 (FIG. 1) may have Velcro® or hook and loop tape attached to every part of the harness so that the pouches can be attached anywhere on the harness. For example, drain bulbs for a mastectomy may be placed at a higher level on the harness than drain bulbs for an abdominoplasty. In other embodiments, the pouches 124 (FIG. 1) can be attached with Velcro® or hook and loop tape for ease of removal, or they may be permanently attached, attached with snaps, tied on with a strap of fabric, or attached through a loop on the harness 100 (FIG. 1) itself.

The vertical strap that loops around the neck, in still other embodiments, may have padding added for comfort. There may also be padding at the shoulder area of the suspender-like straps. The belt strap may contain extension straps attached so that pouches can be attached at an even lower lever for other abdominal surgeries, e.g. colostomies.

While the principles of the disclosure have been described above in connection with specific apparatuses and methods, it is to be clearly understood that this description is made only by way of example and not as limitation on the scope of the disclosure. The invention is as presented in the following claims.

What is claimed is:

1. A harness for holding one or more drain receptacles that drain fluids from a patient following surgery, the harness comprising:
a first member, the first member held circumferentially around the torso of the patient,
a second member having a first end and a second end, the first end attached substantially perpendicular to the first member, the second member extending superiorly along an anterior portion of the patient, the second member held circumferentially along the posterior of a neck of the patient, the second member extending inferiorly from the neck of the patient to the first member, the second end of the first member attached substantially perpendicular to the first member; and
one or more holders, the one or more holders attached to the first member, the one or more holders containing the one or more drain receptacles.

2. The harness as defined in claim 1, wherein the one or more holders attached to the second member.

3. The harness as defined in claim 1, wherein the first member and the second member are straps.

4. The harness as defined in claim 3, wherein the straps are polypropylene webbing.

5. The harness as defined in claim 1, wherein the first member comprises:
a first end; and
a second end, wherein the first end and the second end are connected with a hook and loop fastener.

6. The harness as defined in claim 1, further comprising a third member, the third member having a first end and a second end, the first end attached substantially perpendicular to the second member as the second member extends superiorly between the first end and the neck of the patient, the second end attached substantially perpendicular to the second member as the second member extends inferiorly between the neck of the patient and the second end of the second member.

7. The harness as defined in claim 1, wherein the first end of the second member is detachably connected to the first member, and the second end of the second member is detachably connected to the first member.

8. The harness as defined in claim 7, wherein the first end and the second end are detachably connected with a hook and loop fastener.

9. The harness as defined in claim 1, wherein the one or more holders comprise:
an attachment member, the attachment member forming an opening, the attachment member attached to the first member; and
a pouch, the pouch attached to the attachment member as to form a container for the drain receptacle.

10. The harness as defined in claim 9, wherein the pouch is made from a mesh material.

11. The harness as defined in claim 10, wherein the mesh material is water resistant.

12. The harness as defined in claim 9, wherein the attachment member is detachably connected to the first member with a hook and loop fastener.

13. A harness for holding one or more drain receptacles that drain fluids from a patient following surgery, the harness comprising:
a first strap, worn circumferentially around the torso of the patient;
at least one other strap, the at least one other strap connected by a first end to the first strap and connected by a second end to the first strap, the first end and the second end connected to the first strap at a predetermined distance, the at least one other strap extending over the torso of the patient; and
one or more containers, the one or more containers detachably connected to the first strap or second strap, the one or more containers having a first end forming an opening, the one or more containers comprising a pouch attached to the first end, the opening large enough to accept a drain receptacle into the pouch.

14. The harness as defined in claim 13, wherein the one or more containers are detachably connected by one or more fasteners to include a hook and loop fastener, a button, a snap, or an adhesive.

15. The harness as defined in claim 13, further comprising:
an extension strap, the extension strap having a first end detachably connected to the first strap or the second strap, the second end attached to a container.

16. The harness as defined in claim 13, wherein the one or more other straps are arranged in one configuration from a group consisting of: over one or more shoulders of the patient, crossed in an anterior position on the patient, and crossed in a posterior position on the patient.

17. A method for wearing a harness for holding one or more drain receptacles that drain fluids from a patient following surgery, the method comprising:
placing a neck strap over neck;
pulling a belt strap around torso;
connecting the ends of the belt;
connecting a chest strap to the neck strap; and
attaching one or more pouches to the harness.

18. The method as defined in claim 17, further comprising:
placing one or more surgical drain receptacles in the one or more pouches;
taking off clothes underneath the harness;
bathing with the harness; and
reversing the connecting the chest strap, connecting the ends of the belt, pulling the belt strap around the torso, and placing the neck strap over the neck to take off the harness.

19. The method as defined in claim 18, wherein the patient can wash under the harness by detaching one or more fasteners while bathing.

20. The method as defined in claim 17, wherein one or more of the pouches is attached with an extension strap.
